(19) United States
(12) Patent Application Publication
(10) Pub. No.: US 2019/0357825 A1
(43) Pub. Date: Nov. 28, 2019

(54) OPTICAL SENSOR

(71) Applicant: Oxford University Innovation Limited, Oxford (GB)

(72) Inventors: Rongsheng CHEN, Faringdon (Oxfordshire) (GB); Clive HAHN, Radley (Oxfordshire) (GB); Andrew FARMERY, Oxford (Oxfordshire) (GB); Giovanni FIORONI, Gubbio (IT)

(21) Appl. No.: 16/469,724

(22) PCT Filed: Dec. 15, 2017
(86) PCT No.: PCT/GB2017/053769

§ 371 Date: Jun. 14, 2019

(30) Foreign Application Priority Data
Dec. 16, 2016 (GB) 1621481.9

(51) Int. Cl. A61B 5/1455; G01N 21/64

(52) U.S. Cl. CPC A61B 5/14556; G01N 21/6428; G01N 2201/023; G01N 2021/6432; G01N 2201/088; G01N 21/6408

(57) ABSTRACT

The present invention provides a component for use in an optical sensor, said component comprising a substrate, a surface of the substrate being coated with a layer of a composition comprising: (i) carbon nano-tubes; (ii) an optically-active substance and (iii) a matrix material.

The present invention relates to sensors, e.g. for measuring the concentration of analytes such as oxygen or parameters such as temperature or pressure, particularly optical sensing matrices for liquid and gas phase measurement in medical, biomedical and industrial applications with a rapid response time.

Fast, reliable and accurate sensors are important for many analytical applications in industrial, biomedical and clinical areas. Oxygen sensors are of particular interest in medical and clinical applications, providing measurement of the rate of oxygen consumption by patients, the oxygen partial pressure in the inspired and expired gas of patients undergoing anaesthesia, or in the critical care setting.

The value of measuring rapid oxygen partial pressure (pO₂) oscillations in arterial blood, which may occur on a breath to breath basis in patients with acute lung injury, is becoming of increasing interest. In the sick diseased lung, the alveolar units may start to collapse in expiration and re-open in inspiration. This process, called cyclical atelectasis (CA), causes pO₂ in the arterial blood to oscillate on a breath-by-breath basis. This oscillatory pO₂ signal in arterial blood can be used by the clinician to guide adjustment the ventilator settings to moderate the atelectasis process itself. Therefore, there is a need to measure these intra-breath pO₂ oscillations on-line and in real time with an oxygen sensing device that can be fitted into a human artery in clinical practice. Ideally, the pO₂ measurement range should be from 5 kPa to 60 kPa for such uses.

Conventional electrochemical sensors are relatively slow, often with up to 60 s response times. Although both mass spectrometry and paramagnetic devices have potentially much faster response times of 100-500 ms, they are both bulky and costly and their ultimate response times are limited by gas transport, sampling and signal processing issues. They also tend to be restricted to measurements in the gas phase only.

Optical oxygen sensors based on luminescence quenching offer many advantages over the above and the traditional Clark electrode, such as cost effectiveness, immunity from electromagnetic interference, and due to the fact that they do not consume the oxygen they are measuring. The principle of the optical oxygen sensor is based on the oxygen quenching effect on luminescent light emitted from luminophores that are immobilised in a “sensing matrix”. A short pulse of excitation light from a LED is transmitted along a fibre optic light guide to excite a luminescent dye that is immobilised in the sensing matrix at the sensor tip.

The resulting emission of luminescent light, quenched by the presence of oxygen molecules, travels back up the fibre and is detected by a detector. The lifetime and intensity of emitted fluorescence are inversely proportional to the concentration of gaseous or dissolved oxygen according to the Stern-Volmer relation. These types of optical sensor are both sensitive and stable at low oxygen tensions making them ideally suited to oxygen measurements within the physiological range in biomedical applications. However, the response time of most luminescence based oxygen sensors is reported as being of the order of several seconds. This is thought to be due to low permeability of oxygen in the sensing matrix and the thickness of the sensing matrix in which the oxygen sensitive luminescent dye is immobilized. This makes these sensors less suited to those biomedical applications which require a fast dynamic response, such as breath-by-breath gas or blood analysis.

Many materials have been used in the matrix, including silicone rubbers, silica gels, sol-gels, and polymers. Most of these materials were chosen because they have a high oxygen permeability, good mechanical and chemical stability and superior optical clarity. The response times of current fibre optic oxygen sensors are typically reported to be between 1 and 30 seconds. In principle, the response time of the sensors can be markedly improved by using a material with high oxygen diffusivity or modifying the geometrical properties of the matrix. For example, a thinner sensing film typically produces a more rapid response, but there is a trade-off between an improved response time and reduced luminescence and signal intensity, the latter being the inevitable consequence of the low efficiency of the luminescent excitation due to the thinner sensing film. In order to get a high enough luminescent intensity, the diameter of the optical fibre currently needs to be over 400 μm, with some up to 1000 μm. Fibres of such diameters are unsuitable for use as an intravascular sensor.

Furthermore, when working in a hostile environment such as arterial blood, very thin polymer sensing films are easily damaged and degraded. There thus exists a need for means for sensing analytes such as oxygen with rapid response times and the required sensitivity, while also being of sufficient size and robustness to be suited to in vivo applications.

The present inventors have made the surprising finding that the time response of optical sensors can be significantly improved by including carbon nano-tubes (CNTs) in the sensing matrix, without any loss of sensitivity. This improvement in time response is achievable with sensing films that are sufficiently robust to remain stable in hostile environments such as arterial blood, yet sufficiently small and sensitive for in vivo applications.

Thus, viewed from a first aspect, the present invention provides a component for use in an optical sensor, said component comprising a substrate, a surface of the substrate being coated with a layer/coating of, or comprising, a composition comprising:
(i) carbon nano-tubes;
(ii) an optically-active substance and
(iii) a matrix material.

Optical sensors comprising the components, coated substrates and compositions herein described form a further aspect of the invention.

The sensor or component (e.g. sensor tip) for use in an optical measurement method as described herein may be applied to the measurement of oxygen, however the invention also applies to the measurement of the presence or concentration of other analytes (such as glucose or pH, i.e. H⁺ concentration) or parameters (such as temperature or pressure) which may be measured using optical methods. Preferably the analyte is oxygen (i.e. the sensor is an optical oxygen sensor) and/or the sample is blood, especially preferably, the sensor is an in vivo blood oxygen sensor. Unless specified otherwise, references herein to “analytes” are intended to include parameters such as temperature and pressure.

The sensor or component thereof (e.g. sensor tip) comprises a substrate coated with a composition comprising (i) carbon nano-tubes; (ii) an optically-active substance and (iii) a matrix material. The composition and coated substrate are also novel, and thus form a further aspect of the present invention. Use of a composition, component or coated substrate as described herein in measurement of an analyte or in the production of a sensor (e.g. an optical sensor) or sensor component forms a further aspect of the present invention. Use of a composition, coated substrate or component in an optical sensor forms a further aspect of the present invention.

Thus, in a further aspect, the invention provides a composition comprising:

(i) carbon nano-tubes;

(ii) an optically-active substance and

(iii) a matrix material.

The composition may be viewed as an oxygen-sensitive composition, or a composition for use in oxygen sensing.

Preferably the optically-active substance is a luminophore, especially a fluorophore and/or the matrix material is or comprises a polymer. Although the invention will primarily be described in relation to optical fibre substrates, it is equally applicable to other substrates known in the art, e.g. plates, planar waveguides etc.

The matrix material, in combination with carbon nano-tubes and optically-active substance, can act as a “sensing matrix”. The compositions herein described can be used as a coating in optical measurement devices, e.g. as a sensing matrix for optical sensors. A sensing matrix may be produced by providing a substrate with a layer of (or comprising) the composition of the present invention. Thus, viewed from a further aspect, the present invention provides a coated substrate (preferably an optical fibre), wherein a surface of the substrate is coated with (e.g., a coating or a layer of or comprising) a composition as herein described. Also provided is the use of a composition as herein described as a coating for a substrate and a method for producing a coated substrate, said method comprising coating a surface of said substrate with a composition (e.g. a layer or coating comprising, or of, said composition) as herein described. Also provided is an optical sensor, or component thereof, e.g. sensor tip, comprising a substrate, wherein a surface of the substrate is coated with a composition as herein described. Typically, the component is a sensor tip, a probe, or part thereof. In one aspect, the composition or coated substrate is, or forms part of a sensor tip, e.g. for optical oxygen measurement.

In a further aspect, the invention provides a process for manufacturing a sensor, component or coated substrate (e.g. as described herein), comprising the step of applying a composition as herein described to a surface of a substrate.

The compositions, sensors, components or coated substrates of the present invention, e.g. an optical fibre coated with the composition as herein described, can be used in methods of measurement of analytes such as oxygen, glucose or hydrogen ions and parameters such as temperature and pressure. Thus, viewed from a further aspect, the invention provides a method for measuring a parameter, or the presence or concentration of an analyte, for example dissolved or gaseous oxygen, in a sample, said method comprising using a composition, substrate, sensor or component as herein described. Typically, the measurement method comprises applying a sensor or component as herein described to a sample, supplying light to the optically-active substance via the substrate (e.g. optical fibre), measuring the optical output (e.g. emitted light) of the optically-active substance and using the result to calculate the concentration of the analyte (e.g. using the Stern-Volmer relation) or the parameter.

The supply of light is a pulse of excitation light, e.g. from a LED, which excites the optically-active substance (such as a luminophore) in the sensor tip. For oxygen measurement, the resulting emission of luminescent light, quenched by the presence of oxygen molecules, travels through the substrate (e.g. along an optical fibre) and is detected by a detector. The lifetime and intensity of emitted light are inversely proportional to the concentration of gaseous or dissolved oxygen according to the Stern-Volmer relation.

The compositions of the present invention have been evaluated for measurement of oxygen in a gaseous environment and the results showed that the time response of the sensors is improved (in comparison to a polymer matrix without CNTs) by addition of CNTs into the sensing matrix, whilst the sensitivity of the sensors was unchanged. The time response of the sensors can be less than 100 ms, even when the thickness of the sensing matrix was thicker than 1 μm. Also, the robustness of the sensing film was much improved. The present invention thus achieves rapid oxygen sensing with thicker films (e.g. >1 μm) than those used for current rapid sensors. Although the films may be slightly thicker, the sensitivity is maintained and thus the fibre can be of a sufficiently small diameter to be used in vivo. The problem of the afore-mentioned “trade-off” between an improved response time and reduced luminescence and signal intensity (i.e. reduced sensitivity) is therefore solved by the present invention. Moreover, as the CNTs are simple and inexpensive to incorporate into a polymer matrix, the sensor tip can be formed entirely from biocompatible polymers. The sensing matrix of the present invention has also been found to function without contributing to clotting.

Without wishing to be bound by theory, it is thought that the presence of the CNTs will increase the free volume of the matrix, therefore the permeability (or diffusivity and the solubility) of the matrix is increased. This allows fluid analytes, e.g. gases such as oxygen, to diffuse in and out of the matrix material (i.e. increasing oxygen permeability), thus interacting with the optically-active substance. Thus, the fluid diffusion process saturates more rapidly compared to the macro scale resulting in improved sensor response time. The CNTs may therefore be acting as a nano-filler to form a nano-composite which increases the permeability of the oxygen sensing matrix. It is thought that the sensitivity of the sensors is not affected because some of the analyte, e.g. oxygen molecules, may be trapped inside of the CNTs and does not interact with the fluorophore that is outside of the CNTs. A further aspect of the present invention is therefore the use of CNTs to improve the response time of an optical sensor.

The carbon nano-tubes of the present invention may be single-walled (SWCNTs), double-walled (DWCNTs) or multi-walled (MWCNTs). Single-walled carbon nano-tubes are preferred. Typical dimensions are 0.5 to 15 nm, preferably 1 to 10 nm in diameter with an average length of 1 to 50 μm, preferably 5 to 30 μm. The CNTs may be sonicated prior to being incorporated into the composition of the invention. Sonication may break up the bulk of the CNTs, enabling them to disperse throughout the matrix material more effectively.

The optically-active substance can be any substance which has optical properties that are dependent on the concentration of an analyte. Examples are dyes, luminophores, phosphors and fluorophores. The optically-active material could be such that it is luminescent and the measurements taken are concerned with, for example, the rate of decay of the luminescent effect, which varies in accordance with the concentration of the assay substance. Alternatively, the optically-active substance could be one that has light absorption characteristics that vary dependent on the concentration of an analyte, e.g. a colorimetric measurement may be taken to measure the analyte concentration. Preferably, e.g. for oxygen measurement, the optically-active substance is a luminophore, particularly a fluorophore, whose emission upon excitation is quenched by the presence of oxygen molecules.

Ideally, the substance has a long fluorescence time. Preferably it is non-toxic, especially for in vivo applications.

Any substance suitable for optical measurements may be used as the optically-active substance of the invention, for example, ruthenium, palladium and platinum complexes, e.g. Ru(phen), palladium tetrakis pentrafluoropheny porphine (PdTFPP), platinum tetrakis pentrafluoropheny porphine (PtTFPP) and Platinum-Octaethyl-Porphyrin (PtOEP) are suitable for optical oxygen measurements. Platinum (II) complexes are particularly preferred. Platinum-Octaethyl-Porphyrin (PtOEP) is especially preferred as the optically-active substance of the invention.

Typically, the fluorescence emitted from the fluorophore is in the range of 550 nm-700 nm. Advantageously, at the excitation wavelengths used (e.g. a peak wavelength of around 400 nm to 500 nm), the CNTs themselves do not fluoresce.

Suitable matrix materials include silicone rubbers, silica gels, sol-gels, and polymers. Polymers, i.e. polymeric matrices, are especially preferred.

The matrix material which is combined with the carbon nano-tubes and an optically-active substance to form the sensing matrix of the invention may be any suitable material which is permeable to the analyte to be measured, has good mechanical and chemical stability and adequate optical clarity. In most polymer materials, the larger pendant groups prevent the polymer chains from closing and packing together, resulting in a greater proportion of free volume within the polymer itself. Consequently, polymers with larger pendant groups have a higher oxygen solubility and diffusion coefficient. Therefore, sensors using larger pendant group polymers as the matrix material to immobilize the optically-active substance can have higher sensitivity and faster time-response.

The inventors have found that different polymer materials provide different time responses and different sensitivities. For clinical applications of oxygen measurement, the solubility of oxygen in the matrix material should be low. This property is considered to be important since it reduces the capacity of the matrix to act as a reservoir of “oxygen,” and so reduces the so-called “memory effect” of the sensor which is caused by oxygen continuing to dissolve into or evolve from the reservoir after the ambient has abruptly risen or fallen, respectively. Matrix materials with low solubility for the analyte are thus preferable.

Suitable polymers include those used in existing optical measurement matrices, such as acrylate polymers. Specific examples include poly(cyclohexyl methacrylate) (PCMA), poly(I-menthyl methacrylate) (PMtMA); poly(4-methyl-1-pentene); 3,3,3-trifluoropyltrimethoxysilane (TEPTriMOS); n-propyltrimethoxysilane (n-propyl-TriMOS); poly(methyl methacrylate) (PMMA); poly(ethyl methacrylate); PEMA and poly(propyl methacrylate) PPMA. PMMA, PPMA and PEMA are particularly preferred.

PEMA has been found to be especially preferred. PEMA has high oxygen diffusivity and solubility and thus enables high oxygen sensitivity.

When both the substrate and the matrix are polymeric, the matrix can comprise (i.e. comprise, consist essentially of, or consist of) the same polymer(s) as, or different polymer(s) to the substrate, e.g. optical fibre. In such cases, the polymeric matrix may comprise any suitable polymeric material provided it is compatible with the substrate and will adequately adhere to a surface thereof. The matrix and/or substrate may consist of one or more types of polymer, however, for ease of manufacture they may each consist of the same polymer.

The composition of the present invention may or may not further comprise a solvent. In the composition, the carbon nano-tubes and optically-active substance may be dispersed in the matrix material. Alternatively, when a solvent is present, all three components may be dispersed in said solvent. Suitable solvents include toluene, ethanol, acetone, dichloromethane, especially dichloromethane.

The composition may be produced by mixing the various components in any convenient order, in the presence of a solvent if necessary. Preferably, the optically-active substance and the matrix material (added in any order) are mixed together in a solvent and the CNTs then added. Alternatively, the CNTs can be added at the same time as the other components.

In a preferred aspect, the CNTs and optically-active substance are dispersed in the matrix material (preferably with a solvent also present), forming a mixture, e.g. a suspension or solution. Such a mixture can be made by mixing the required amount of CNTs and optically-active substance in the matrix material. The mixture can be formed through any suitable technique, including mechanical mixing, shear mixing, magnetic mixing, ultra-sonication or a combination of all of these methods, until adequate dispersion of CNTs and optically-active substance in the matrix material (and/or solvent) is achieved.

To form a coating/layer, the composition may be applied to the substrate by any suitable means, e.g. standard thin film coating processes. Suitable techniques also include painting, spraying and spreading onto a surface of the substrate. In order to adjust the viscosity of the layer in order to facilitate application, further components such as solvents or thickening agents may be applied. Most preferably, the substrate is dipped into the composition, removed and then allowed to dry (e.g. to evaporate any solvent) to form the coating. The surface of the substrate to be coated with the composition may be pre-treated before the coating is applied, e.g. to improve adherence of the coating. Such pre-treatment steps may include application of one or more base coats, removal of a portion of the substrate, e.g. “decladding” of a substrate such as an optical fibre surface and/or cleaning of the surface, e.g. with a solvent such as iso-propyl alcohol.

The composition (particularly when present on a substrate, i.e. as the coating or layer described herein) may be referred to as a sensing matrix. The coating may be thus be regarded as a nano-composite coating, layer or film. The present invention thus provides a nano-composite sensing matrix, composition, layer or film for an optical sensor, comprising:

(i) carbon nano-tubes;

(ii) an optically-active substance and

(iii) a matrix material.

The carbon nano-tubes and optically-active substance are thus in combination with the matrix material. In the coated substrate of the invention, the carbon nano-tubes and optically-active substance are dispersed or held, in or on, the matrix material.

Preferably, the carbon nano-tubes and optically-active substance are dispersed in the matrix material. Optionally, other materials may be present in the composition or layer/coating, however, preferably the composition or layer/coating consists of or consists essentially of the carbon nano-tubes, the optically-active substance and the matrix material. The term “dispersed in” is intended to encompass both the situation where all, or substantially all, of the carbon nano-tubes and/or optically-active substance are surrounded by matrix material; and the situation where some carbon nano-tubes and/or optically-active substance are only partially surrounded by matrix, e.g. where they are embedded in said matrix, but at least partially exposed at a surface of the layer.

Most preferably, the carbon nano-tubes and/or optically-active substance are substantially encompassed by matrix material. Especially preferably, the carbon nano-tubes and/or optically-active substance are evenly dispersed throughout the matrix material.

By their nature, the CNTs will be present as discrete particles/fibres (although they may be aggregated). The optically-active substance is typically bonded to or blended with the polymer.

The amount of optically-active substance present in relation to the matrix material may be similar to that used in prior art sensing matrices. For example, weight ratios of optically-active substance to matrix material may be in the region of 1:500 to 1:50, e.g. around 1:100.

Weight ratios of CNTs to matrix material may be in the region of 1:50 to 1:1, e.g. around 1:5. Expressed as a weight percentage (wt. %) of the total weight of the composition (i.e. matrix material+optically-active substance+CNTs), the amount of CNTs in the composition is in the range of 0.1 to 30 wt %, e.g. 0.1 to 20 wt. %, e.g. 0.5 to 10 wt. % or 10 to 20 wt. %, especially 1 to 5 wt. %.

Using sonication to break up the CNTs, prior to their combination with the matrix material, may enable lower amounts to be used.

The nature of the substrate to which the coating is applied will depend on the sample to be tested. Substrates used in prior art methods may be used. For optical measurement methods, the substrate should be capable of transmitting light and thus preferred substrates are plates, optical waveguides (e.g. planar waveguides) and optical fibres. Optical fibres are particularly preferred.

Optical fibres known in the art are suitable, e.g. silica fibres or polymeric optical fibres (i.e. fibres formed from or comprising one or more polymers). Silica/glass fibres may be less preferred, e.g. for in vivo applications, due to their fragility. Polymeric optical fibres may be formed from polymers such as those described herein for use as the matrix material. For ease of manufacture, the matrix material may be the same polymer as the optical fibre. Especially preferred optical fibres are PMMA fibres.

Typical fibre diameters are in the range of 100 to 1000 μm, e.g. 300 to 400 μm, especially around 500 μm.

The present compositions and coatings are applicable to a variety of sensors and sensor components known in the art. For example, the present inventors have found that a “cylindrical-core” design can improve the time response of fibre optic oxygen sensors. By using, as the matrix material, a material with a higher refractive index than the optical fibre, the fibre sensing element becomes a cylindrical-core waveguide and the most of the excitation light from the guiding fibre is coupled into the cylindrical-core waveguide. Therefore the excitation light will interact strongly with the luminophore in the sensing matrix on the fibre even with a very thin layer. A further suitable type of sensor is one with a tapered tip, preferably with the coating on the tapered section.

The coating should be applied on a surface of the substrate which comes into contact with the sample during the analysis technique. When the substrate is an optical fibre, the coating is therefore typically applied on or around the fibre tip, i.e. the end of the fibre furthest away from the excitation light source.

The surface to be coated is therefore preferably the outer surface of an optical fibre, particularly proximate to the fibre tip, optionally including the terminal cross sectional area.

The surface texture of the coating/layer of composition on the substrate may be approximately homogenous overall, e.g. characterised by a definable texture depth or other texture parameter. It is also preferable for the coating to have a repeatable thickness, e.g. when coating multiple components that must be coated within certain tolerance ranges. Preferably, the coating has a substantially constant thickness. Typical average thicknesses for the coating are 0.1 μm to 20 μm, e.g. 0.5 μm to 10 μm, especially 0.5 μm to 1 μm or 1 μm to 5 μm. The coating is preferably at least substantially continuous across its area.

The area of substrate covered by the coating will depend on the intended use. For industrial and environmental applications, larger areas will be covered than for clinical, especially in vivo, uses. The coating may cover up to 100%, preferably greater than 10%, e.g. greater than 50% of the area of the sensor which is exposed to the sample. For optical oxygen sensing, a section of 0.1 to 20 mm in length may be coated, preferably 0.25 to 10 mm, especially 0.50 to 1.50 mm, e.g. around 1 mm. Preferably the coating extends over the entire outer surface of the substrate, e.g. for a cylindrical fibre, round the circumference. For planar substrates, the area covered may be any convenient shape and size and will be apparent to the reader.

The coating layer of composition present on the surface of the substrate may of course comprise one or more coating layers. Furthermore, the coating layer may comprise any further suitable material(s) which would impart the desired properties to the coated substrate. However, it is an advantage of the present disclosure that the matrix material can encapsulate the optically-active substance and CNTs in such a way as to obviate the need for a further coating on the substrate. Nonetheless, further coatings known in the field may be present or added if desired. For example, one or more layers (e.g. a base coat) may be present between the substrate and the coating of the present invention. Examples of such layers are those added to substrates such as fibres during their manufacture (e.g. layers or coatings can form part of the substrate) and may or may not be removed prior to application of the coating layer of the invention. Thus, the layer of composition need not be coated directly onto the substrate, in which case, the component can be viewed as comprising the substrate and composition herein described. Alternatively or additionally, one or more further coatings may be added to the coating of the invention.

The present invention is not limited to clinical use, i.e. it is also applicable to industrial and environmental measurements. The present invention is applicable to measurements in both the gas and liquid phases, for medical, biomedical, environmental (e.g. water samples) and industrial applications (e.g. bioreactors). For medical applications, the subject may be human or animal.

The measurement methods and sensors herein described may be used for the optical measurement of analytes such as oxygen, glucose and hydrogen ions and parameters such as temperature and pressure. In a preferred aspect, the compositions and coatings herein described are for an oxygen sensor, i.e. the analyte is oxygen. For medical/clinical purposes, the measurement may be of oxygen concentration in breath (inspired or expired) or blood (venous or arterial). The measurement methods described herein may be made in vivo or ex vivo. Preferably the sample is blood, especially human blood.

For ex vivo measurement of body fluids, the invention may be applied to a sample that has been removed from the body, e.g. a blood sample. As the invention may be applied to substrates such as thin optical fibres, in vivo methods are also provided.

All references herein to “comprising” should be understood to encompass “including” and “containing” as well as “consisting of” and “consisting essentially of”.

The disclosure of each reference set forth herein is specifically incorporated herein by reference in its entirety.

The invention will now be described in more detail with reference to the accompanying figures, in which:

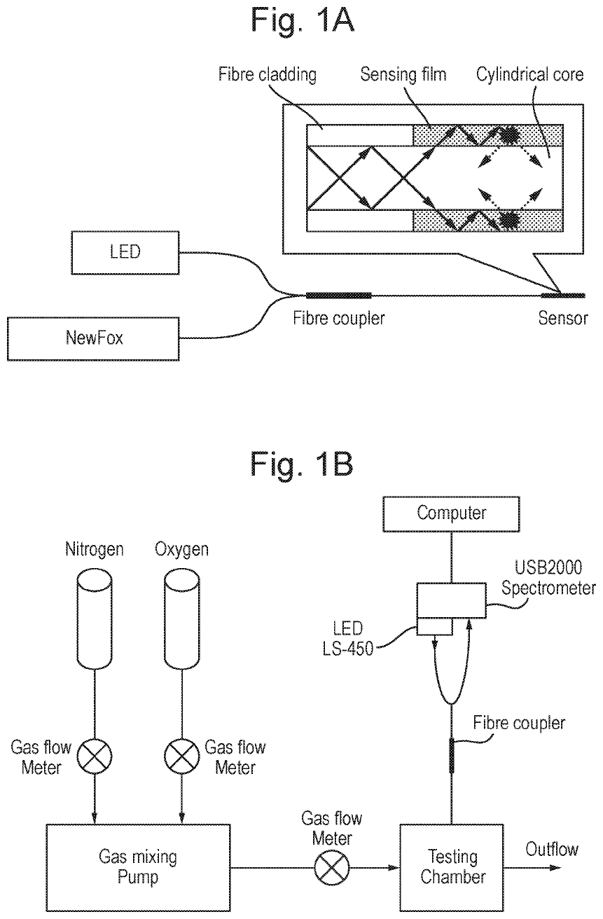

FIG. 1A shows a schematic of a measurement apparatus, focussing on the sensor tip.

FIG. 1B is a schematic illustration of the experimental set-up for evaluating sensor sensitivity in the gas phase.

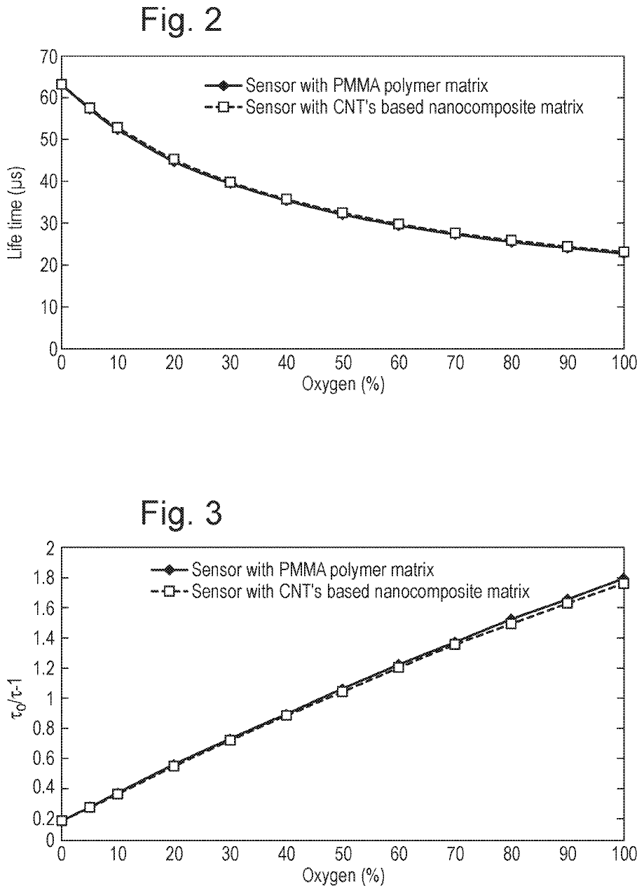

FIG. 2 shows the variations of luminescent life time as a function of oxygen concentration for two fibre optic oxygen sensors with different sensing matrices.

FIG. 3 shows Stern-Volmer plots (representing sensitivity of the sensors) for two fibre optic oxygen sensors with different sensing matrices.

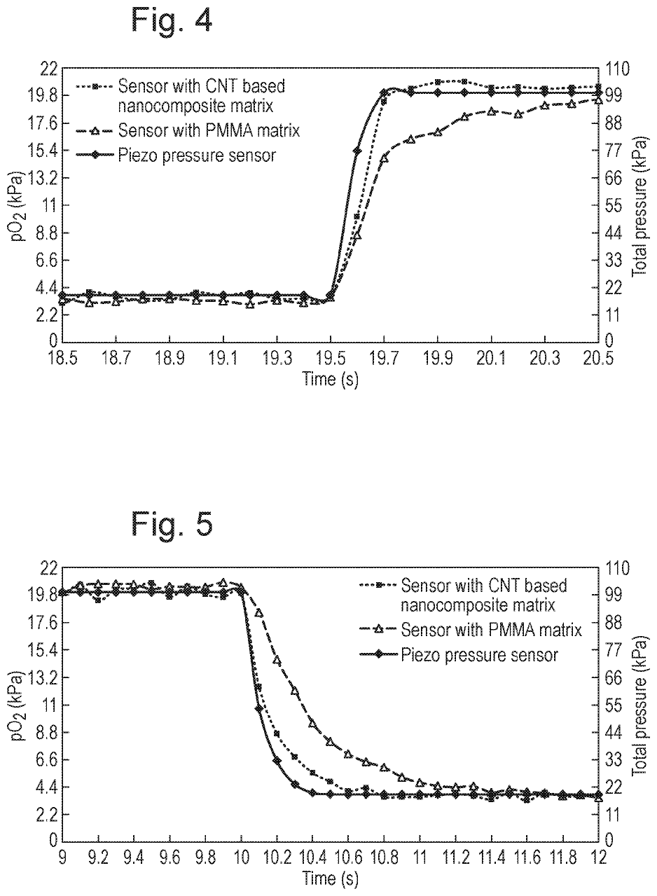

FIG. 4 shows the signal responses from a piezo pressure sensor and the fibre optic sensors to the change in pressure of a test chamber from 19 kPa to 100 kPa and the pO₂ step change from 3 kPa to 21 kPa in the test chamber.

FIG. 5 shows the responses of a piezo pressure sensor and the fibre optic sensors to the change in pressure from 100 kPa to 19 kPa and the pO₂ step change from 21 kPa to 3 kPa in the test chamber.

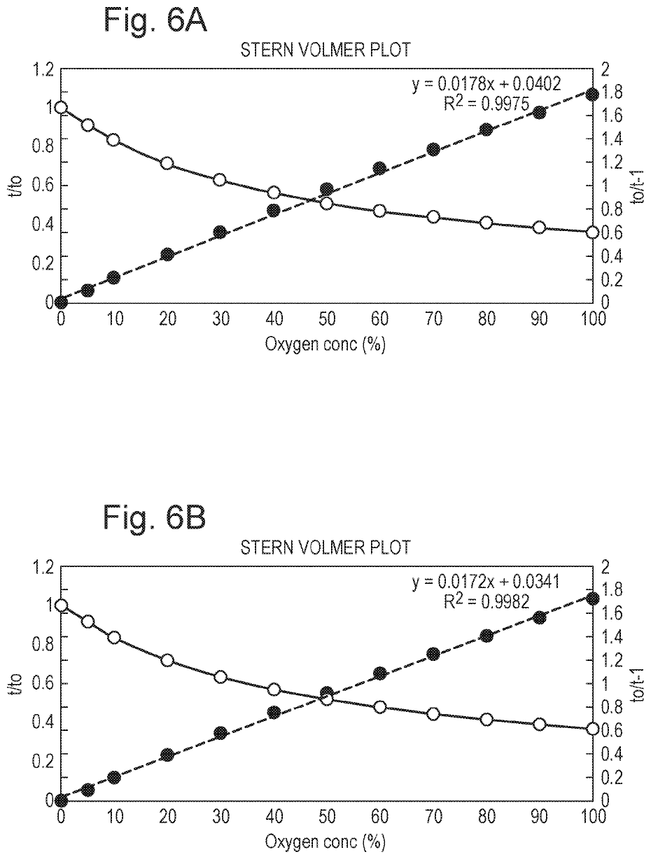

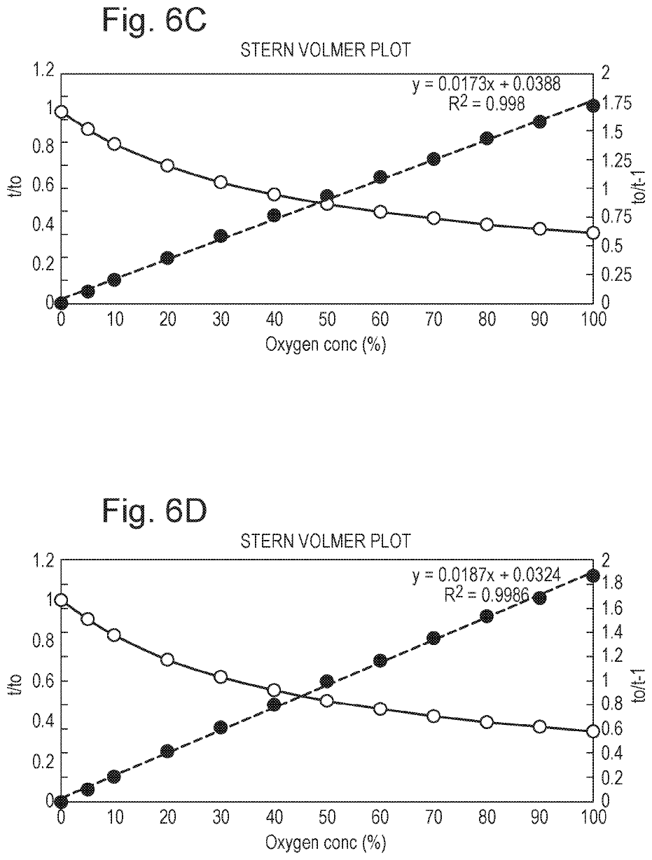

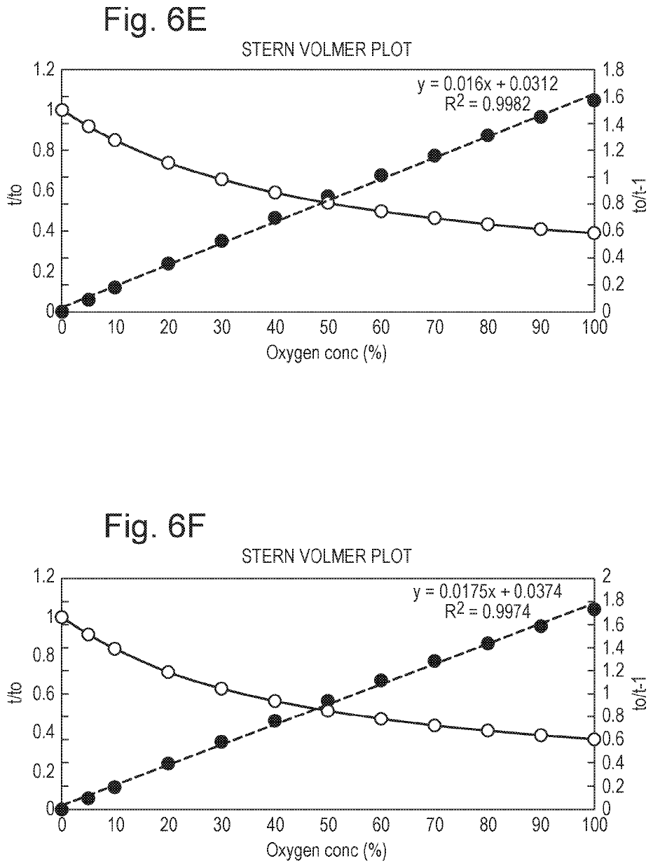

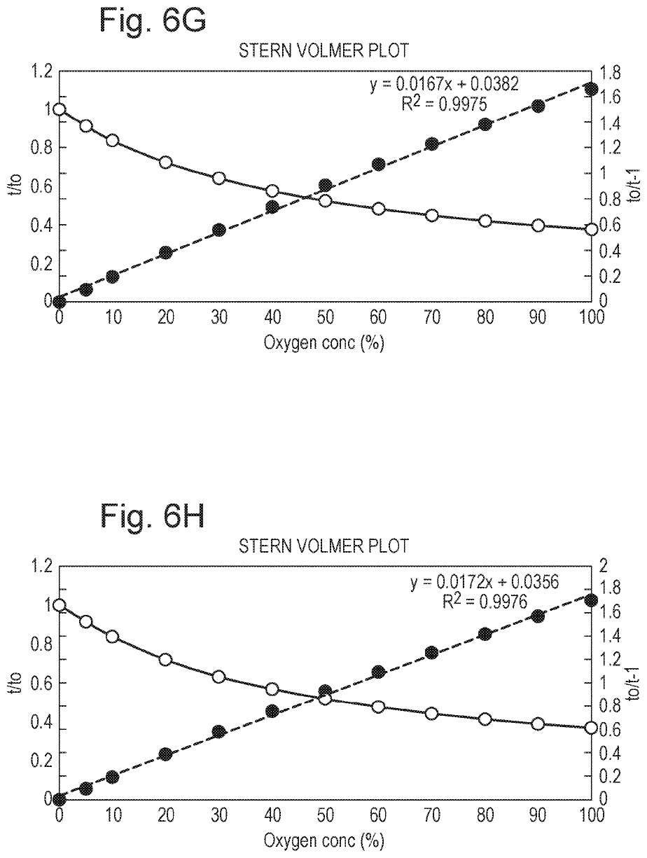

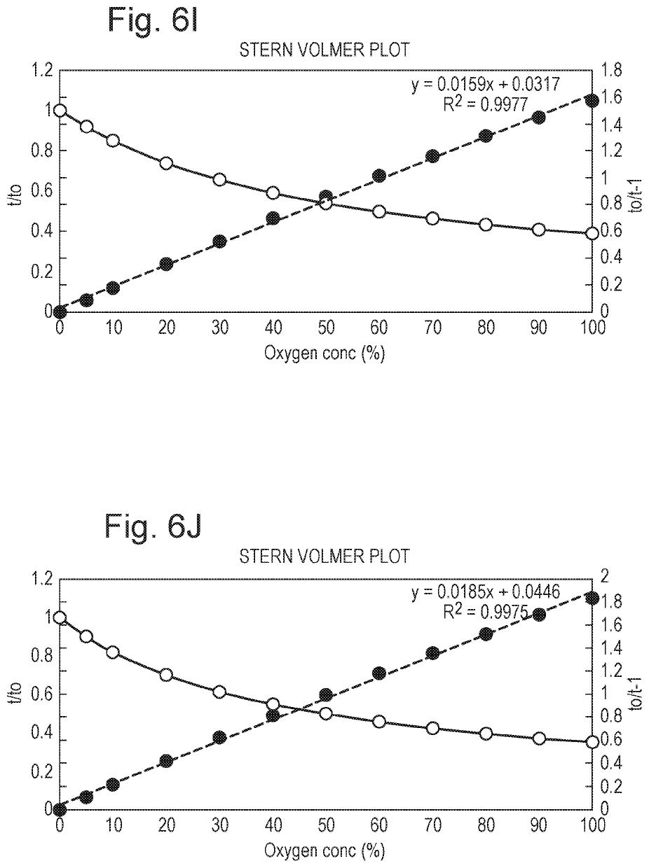

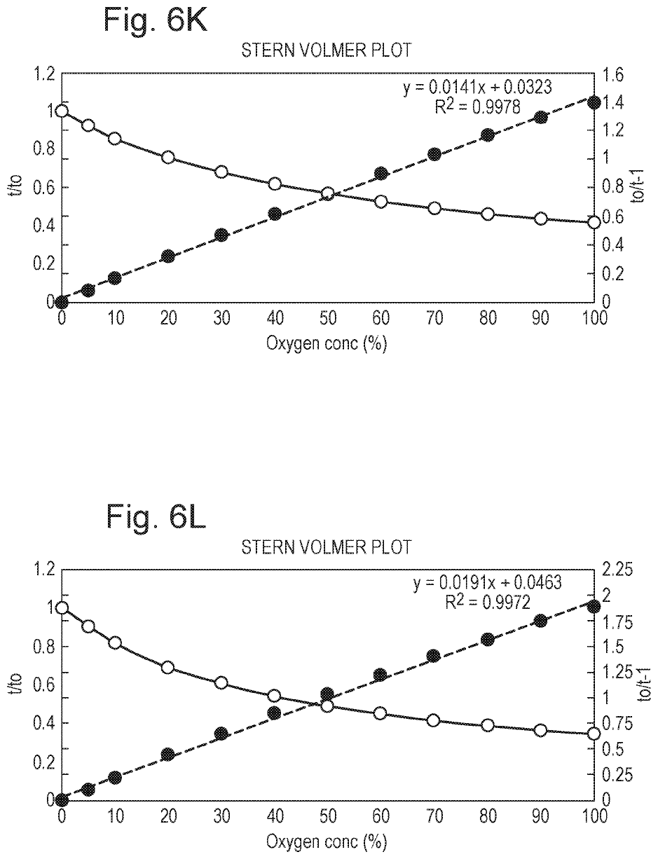

FIG. 6A-L are sensor sensitivity results.

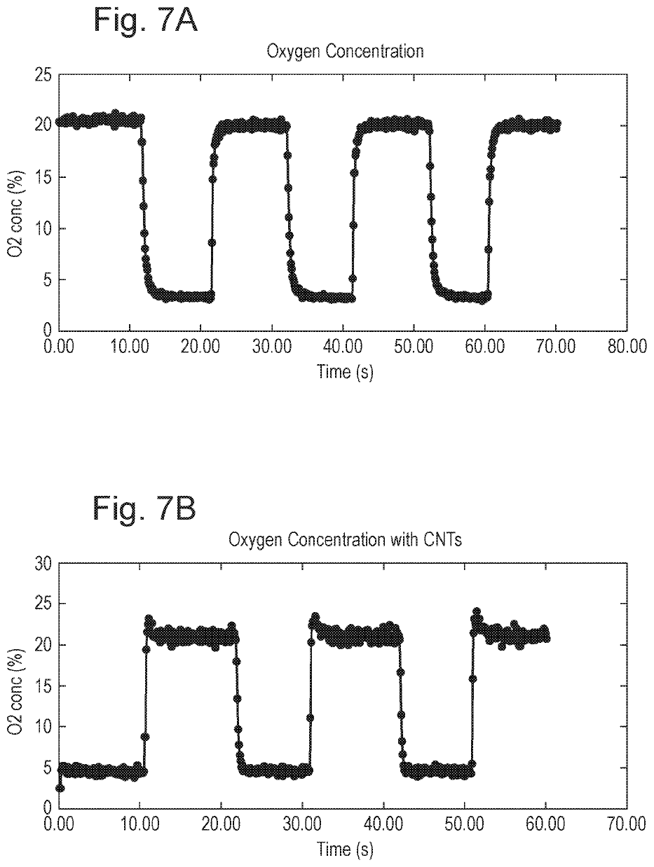

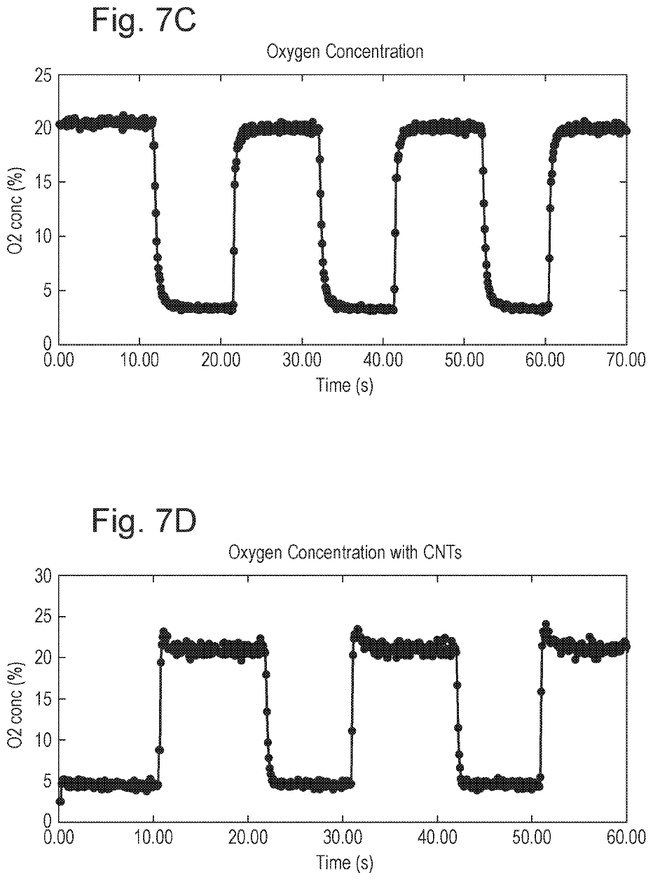

FIG. 7A-D are response time results.

As noted above, FIG. 1A shows an example of a typical measurement set-up according to the present invention. The sensing layer, comprising matrix material, CNTs and optically-active substance, is applied to the substrate by any suitable means. For an optical fibre, the area to be coated may be decladded using standard techniques if desired, e.g. to remove any coating or to prepare the surface such that it has a stronger bond with the sensing layer to be added. For example, FIG. 1A shows a PMMA based polymer (matrix material) sensing film (about 2 μm thickness), containing CNTs and an optically-active substrate (PtOEP). FIG. 1A shows the sensing film, i.e. layer of the composition of the present invention coated at the decladded end section of an optical fibre to form a sensor head or tip. Typically such coatings may be formed by dipping the fibre into a solution of polymer, luminophore and CNTs in a solvent such as dichloromethane.

Prior to the dipping and coating process, the substrate is optically decladded, cleaned and/or dried. The section of the substrate to be coated is then dipped into the luminophore-doped polymer solution and withdrawn from it sufficiently slowly for some of the solution to adhere to the substrate surface. As the solvent evaporates, a sensing film is formed on the substrate. The coated substrate may be dried for some time prior to its use in a measurement method.

The “sensing” layer of composition as herein described should be conveniently placed on the substrate such that it is accessible to the sample in which the analyte, e.g. oxygen, is to be measured. For applications where the sensor is being used as a probe, e.g. for dipping into water or blood samples, or intravenous measurements, the layer will be on or near the part of the substrate that is furthest from the excitation light source, e.g. towards the tip of an optical fibre.

The compositions of the present invention are advantageously applicable to known optical sensors (e.g. by replacing the existing sensing matrix with the layer of the invention) and thus, for measurement of analytes in, e.g., blood (ex vivo and in vivo), breath, industrial fluids and environmental applications, standard procedures and apparatus known in the relevant field can be applied. Due to the improved time response, sensors comprising the compositions of the present invention may be inserted into the blood vessel of a patient for real time in vivo measurements. For intravenous applications, the sensor can be attached to the measurement apparatus using standard connectors.

When the substrate coated with the composition of the invention, e.g. the optical fibre of FIG. 1A is in contact with the sample, e.g. a blood sample, analyte concentration can be measured using standard optical techniques based on luminescence. In the measurement method, a pulse of excitation light, typically from a LED is transmitted through the substrate, e.g. along the optical fibre to the part which is coated with the composition and in contact with the sample. The light excites the luminophore, thus causing it to luminesce. The emission of luminescent light travels back up the fibre and is detected by a detector. The presence of the analyte alters the emission in a way that enables the concentration of analyte to be determined. For example, for oxygen measurement, the emission is quenched in the presence of oxygen molecules such that the lifetime and intensity of the emitted light are inversely proportional to the concentration of gaseous or dissolved oxygen. Using the Stern-Volmer relation, the concentration of oxygen in the sample may be determined.

A measurement apparatus thus typically comprises, in addition to the component or coated substrate of the invention, an excitation light source, e.g. an LED and a detector. In order that the light may pass from the LED to the sensor tip and back to the detector, these may be linked using a Y-type optical fibre coupler such as that shown in FIGS. 1A and 1B. Suitable detectors include fluorescent lifetime measurement systems such as those obtainable from Neofox Ocean Optics.

Measurements may conveniently be carried out at room temperature and atmospheric pressure, e.g. for clinical applications, but the invention is also applicable to non-ambient conditions, such as those encountered in industrial settings.

The oxygen concentration result is typically expressed as pO₂ (e.g. in sensor time response tests in a pressure change chamber).

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLE 1

The influence of adding CNTs into polymer material on the permeability of the polymer and the time response of the oxygen sensors was investigated by using CNTs as nano-filler to form a nano-composite in the oxygen sensing matrix. The performance of the CNT based nano-composite was evaluated by testing the sensitivity and time response of the fibre optic oxygen sensors.

A PMMA based polymer sensing film (about 2 μm thickness), which contained CNTs and PtOEP, was coated at the decladded end section of a silica fibre to form a sensor head. This structure enhances a high luminescence excitation and produces a strong luminescent emission. The luminescent light emitted from the luminophore was collected by the same fibre and its lifetime was measured using a phase measurement system (NeoFox, Ocean optics) in the experimental set-up (see FIG. 1A).

Poly(methyl methacrylate) (PMMA), single-walled CNTs and dichloromethane (as solvent) were obtained from Sigma-Aldrich (USA). The luminophore, Platinum-Octaethyl-Porphyrin (PtOEP), was purchased from Porphyrin (USA). For the preparation of the nano-composite sensing films, two luminophore-doped polymer solutions were made firstly by mixing and dissolving 0.5 mg PtOEP and 50 mg PMMA material in 1 ml dichloromethane, then 10 mg of sonicated CNTs were added to one of polymer solutions to form a CNT-based nano-composite polymer solution. The remaining solution did not contain CNTs. The two polymer solutions were then capped and stirred to ensure complete dissolution of the polymer and the luminophore. Prior to the dipping and form coating process, the 10 mm end section of a pigtailed silica optical fibre (2 m length and 200 μm core diameter) was decladded and cleaned with IPA (iso-propyl alcohol), and dried for 10 minutes. The decladded section of the optical fibre was dipped into the luminophore-doped polymer solutions and then withdrawn from it slowly. An oxygen sensing film was thus formed on the end section of the optical fibre as the solvent quickly evaporated. After coating, the optical fibre was dried for at least 24 hours. The process was carried out at room temperature. In this way a total of six fibre optic oxygen sensors were made. Three had a PMMA polymer sensing film (without CNTs), and the other three had a CNT-based nano-composite sensing film. The sensitivities and time responses of the sensors were evaluated using the method of luminescence life-time measurement.

FIG. 1B shows a schematic illustration of the experimental set-up for evaluating sensor sensitivity in the gas phase. During the experiment, nitrogen and oxygen gas from cylinders were mixed and the mixing ratio was controlled using a precision gas mixing pump (Wostoff, Germany) before flowing into the gas testing chamber. The oxygen concentration was also measured using an oxygen analyzer (Servomex OA570).

During the experiment, the fibre optic oxygen sensor was inserted into the testing chamber and the lifetime of the luminescent light from the sensor head was measured using phase measurement system (NeoFox, Ocean optics). Luminescence excitation of the sensor was provided by a LED (LS-450, Ocean Optics) with a central wavelength of 450 nm attached to a fluorescent lifetime measurement system (Neofox Ocean Optics). The fibre optic oxygen sensing system consisted of a fibre optic oxygen sensor (inserted in the testing chamber) and a Y-type optical fibre coupler. The excitation light from the LED was fed to the sensor, and the emitted luminescent light from the sensor was transmitted to the lifetime measurement system the fibre coupler. The lifetime of the luminescence was measured by the system. The experiment was carried out at room temperature and one atmosphere pressure.

Sensor Response Time

The gas pressure chamber system used in Chen et al., Sensors and Actuators B 222, 531-535, 2016 was used. The test chamber was connected to a second buffer chamber that was continuously evacuated by a vacuum pump (MZ 2C NT, Vacuubrand GMBH, Wertheim, Germany). The chamber had a controlled leak to atmosphere so that by switching the chamber to the buffer chamber or to atmosphere pO₂ in the test chamber could be switched swiftly between any pre-set level between 21 and 0 kPa (vacuum). The dynamic change in total pressure was measured by a Honeywell piezo resistive pressure sensor with a time response of 1 ms (RS Components Ltd, UK). The response time of the oxygen sensor was tested in response to the above near step changes in pO₂.

Sensor Sensitivity Evaluation

FIG. 2 shows the variations of luminescent lifetime as the function of oxygen concentration for the sensors with two different sensing matrices. FIG. 3 shows the Stern-Volmer plots (represented sensitivity of the sensors) of the sensors at room temperature setting, which revealed that the sensors all yield linear Stern-Volmer plots and the sensitivities (τ0/τ100−1, where τ0 and τ100 are the excited state luminescence lifetimes in the absence and presence of oxygen respectively) of the sensors are around 1.75, which correlated to the oxygen concentration changing from 0% to 100%.

Response Time Evaluation

FIG. 4 shows the signal responses from the piezo pressure sensor and the fibre optic oxygen sensors to the change in pressure of test chamber from 19 kPa to 100 kPa and the pO₂ change from about 3 kPa to 21 kPa in the test chamber; FIG. 5 shows the responses of the piezo pressure sensor and the fibre optic oxygen sensors to the change in pressure from 100 kPa to 19 kPa and the change of pO₂ from 21 kPa to about 3 kPa in the test chamber.

The results showed that the response of the sensor was improved by adding CNTs into the polymer sensing matrix and the sensitivity was kept unchanged, which indicates that the CNTs only affect oxygen diffusivity and not solubility of the sensing matrix.

This experiment therefore demonstrates the feasibility of optimizing the time response of silica fibre optic oxygen sensors by using a CNT based nano-composite sensing matrix. The maximum sensitivity factor of the sensors (τ0/τ−1) was approximately 1.75 with a faster response time than that with pure polymer sensing matrix. The CNT nano-composite allows for a thicker matrix film thickness to be used which provides a greater signal to noise ratio and is more physically robust, but without compromising the response time of the sensor.

### EXAMPLE 2

### Sensor Sensitivity Evaluation

Example 1 was repeated (without CNTs), with sensor sensitivity results similar to FIGS. 2 and 3 shown in FIGS. 6A to 6L.

| FIG. | PROBE CODE |
|---|---|
| 6A | O-AK001B-1 |
| 6B | X-AA001B-1 |
| 6C | X-AE001B-1 |
| 6D | X-AC001B-1 |
| 6E | Y-AG001B-1 |
| 6F | Z-AO001B-1 |
| 6G | O-AL001B-2 |
| 6H | O-AN001B-2 |
| 6I | Y-AB001B-2 |
| 6J | Y-AF001B-2 |
| 6K | Z-AD001B-2 |
| 6L | Z-AP001B-2 |

### EXAMPLE 3

### Response Time Evaluation

Example 1 was repeated, with response time results similar to FIGS. 4 and 5 shown in FIGS. 7A-7D. FIGS. 7A (Sensor 1, PMMA only) and 7C (Sensor 3, PMMA only) show results for sensors which contained PMMA, but no CNTs. FIGS. 7B (Sensor 2, PMMA+CNTs) and 7D (Sensor 4, PMMA+CNTs) show results for sensors which contained PMMA and CNTs.

### EXAMPLE 4

### PEMA Matrix

A nanocomposite sensing matrix with PEMA as the polymer was used to fabricate fibre optic oxygen sensors and the time response of the sensors were evaluated in gas phase test chamber.

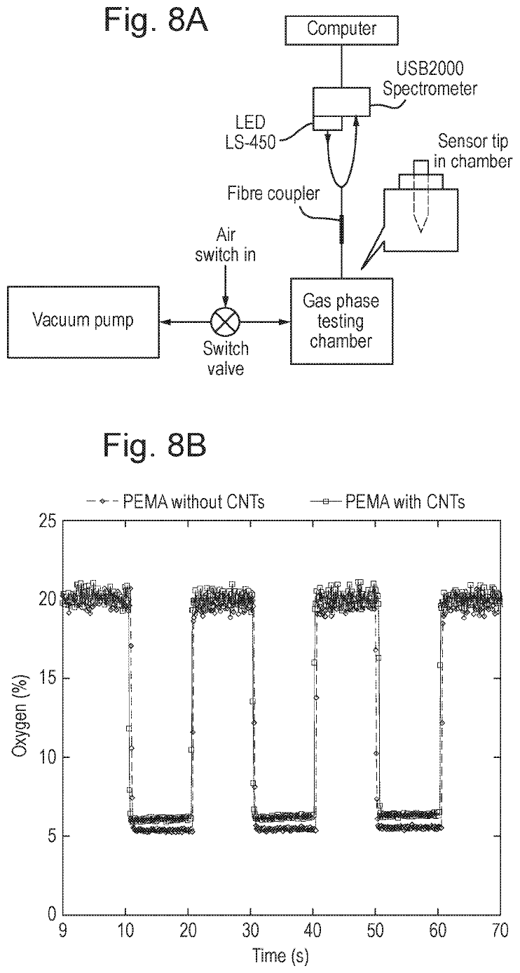

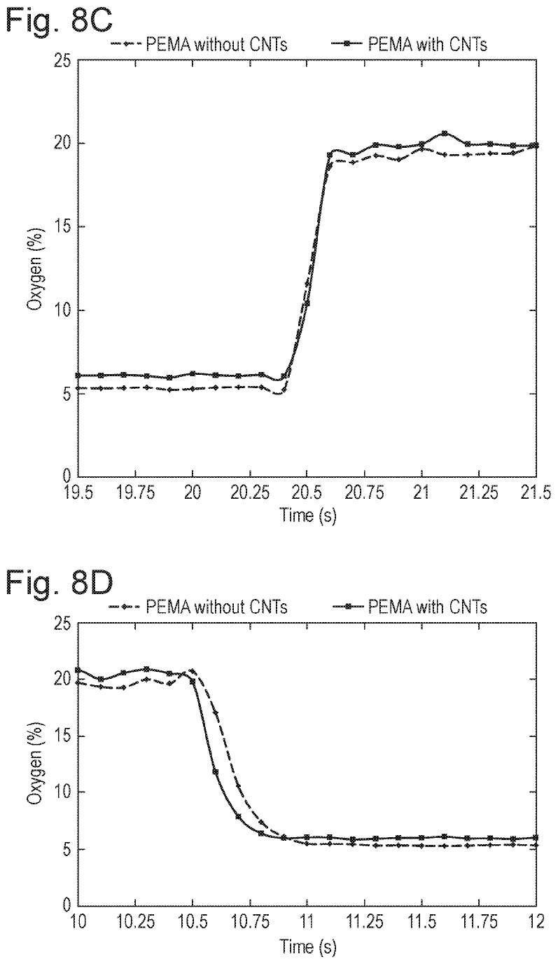

A schematic diagram of the gas phase test system is shown in FIG. 8A. A vacuum pump was used to extract air from the test chamber and an electrical switch valve was used to make a pressure stepping change in test chamber. The oxygen partial pressure (pO₂) in the test chamber was changed with the total pressure step change in the chamber. Two polymer probes (one with PEMA matrix and one with PEMA+CNT matrix) were inserted into the chamber and tested separately. Data was recorded by using Ocean Optics-NewFox with 100 ms sample rate, the modulation frequency was set at 1.46 kHz for the tests.

During the experiments the total pressure in the chamber was changed several times and FIG. 8B shows the pO₂ level changes with total pressure change in the test chamber measured by sensors. FIG. 8C shows the comparison result between the two probes while pressure step increasing. FIG. 8D shows the comparison result between the two probes while pressure step decreasing. The time response of the optical oxygen sensor has thus been shown to be improved by using poly ethyl methacrylate (PEMA) based nanocomposite sensing materials comprising CNTs.

1. A component for use in an optical sensor, said component comprising a substrate, a surface of the substrate being coated with a layer of a composition comprising:
(i) carbon nano-tubes;
(ii) an optically-active substance and
(iii) a matrix material.

2. The component of claim 1 wherein said optically-active substance is a fluorophore.

3. The component of claim 1 or claim 2 wherein said optically-active substance is Platinum-Octaethyl-Porphyrin.

4. The component of any preceding claim wherein said matrix material is a polymer.

5. The component of any preceding claim wherein said composition further comprises a solvent.

6. The component of any preceding claim wherein the weight ratio of carbon nano-tubes to matrix material is 1:50 to 1:1.

7. The component of any preceding claim wherein said substrate is an optical fibre.

8. The component of any preceding claim wherein said substrate is a polymeric optical fibre.

9. The component of any preceding claim wherein said component is a sensor tip, a probe, or part thereof.

10. A composition as described in any one of claims 1 to 6.

11. A coated substrate as described in any one of claims 1 to 8.

12. A method for measuring a parameter or the concentration of an analyte in a sample, said method comprising using a composition, substrate or component as claimed in any one of the preceding claims.

13. The method of claim 12 comprising applying a component as claimed in any one of claims 1 to 9 to a sample, supplying light to the optically-active substrate via the substrate, measuring the optical output of the optically-active substance and using the result to calculate the parameter or the concentration of the analyte.

14. The method of claim 12 or claim 13 wherein the analyte is oxygen.

15. The method of any one of claims 12 to 14 wherein said sample is blood.
